(19) United States
(12) Patent Application Publication
(10) Pub. No.: US 2003/0008312 A1
(43) Pub. Date: Jan. 9, 2003

(54) Provision of DNA investigating tools

(75) Inventors: Peter David Gill, Birmingham (GB); Javaid Iqbal Hussain, Birmingham (GB); Adam Spencer Long, Birmingham (GB); Gillian Tully, Lambeth (GB)

Correspondence Address: MERCHANT & GOULD PC, P.O. BOX 2903, MINNEAPOLIS, MN, 55402-0903, US

(73) Assignee: The Secretary of State for the Home Department, Birmingham (GB)

(21) Appl. No.: 10/174,148

(22) Filed: Jun. 17, 2002

(30) Foreign Application Priority Data
Jun. 15, 2001 (GB) 0114632.3

(51) Int. Cl. C12Q 1/68; C07H 21/04; C12P 19/34

(52) U.S. Cl. 435/6; 435/91.2; 536/24.3

(57) ABSTRACT

A method of evaluating primers for use in the amplification of DNA sequences, a method of producing a mixture of primers for such a purpose and a method of amplifying a plurality of DNA sequences using a mixture of primers is provided in which the evaluation involves performing one or more initial evaluations, obtaining a plurality of SNP sites and related potential primer identities which are passes, generating primers corresponding to those primer identities and conducting a test amplification process using those primers in conjunction with a further evaluation of the results, the pass primers therefrom forming a pool of primer candidates from which one or more primers for use in the amplification of DNA sequences are selected.

The invention provides a faster, cheaper and more versatile technique for developing multiplexes, particularly for forensic science type investigations.

This invention concerns improvements in and relating to the provision of DNA investigating tools, particularly but not exclusively in relation to the provision of suitable targets for investigation and/or suitable primers for investigating those targets and/or suitable multiplexes of primers.

Single nucleotide polymorphisms, SNPs, are of considerable interest to medical investigations and are of increasing interest in forensic investigations. SNPs represent single based locations where variations between the sequence for one being another can occur. An SNP may for instance be the presence of G or C, or of A or T, in the sequence of an individual, with some of the individuals having one of the options, and other individuals having the other options. By considering a large number of such SNPs at different loci, a set of SNP results for an individual can be obtained which is useful for investigative purposes. The results may be compared with the results from another sample, with the statistical occurrence of that set of results within the population as a whole or used in other ways. As each SNP can only vary between one of two options, the substantial number of different locations, generally several hundred loci, need to be investigated to achieve a set of results which is statistically significant in comparisons or other uses for forensic purposes. In this regard, forensic applications are significantly different from medical applications where very rare SNP variations are considered and the presence of even a limited number of SNPs with certain identities can be highly informative on a genetic condition.

Analysing a large number of loci to determine the identity of SNPs on them is a highly time consuming process if all the loci are considered individually and introduce significant compatibility and reliability problems if multiplexes are used to analyse a large number of these loci simultaneously.

The present invention has amongst its aims to provide an improved technique for evaluating SNP targets for consideration in forensic investigations. The present invention has amongst its aims the provision of an improved technique for considering the suitability of primers for adoption in forensic investigations. The present invention has amongst its aims the provision of improved multiplexes involving a plurality of primers for use in forensic investigations.

According to a first aspect of the invention we provide a method of evaluating primers, the primers being for use in the amplification of DNA sequences incorporating one or more single nucleotide polymorphisms, the method including:

selecting a single nucleotide polymorphism site;

generating at least one potential primer identity for amplifying the single nucleotide polymorphism site and performing an evaluation on the potential primer identity and/or the single nucleotide polymorphism site against one or more criteria, the potential primer identity and/or the single nucleotide polymorphism site being deemed to pass or fail the evaluation;

obtaining a plurality of single nucleotide polymorphism sites and related potential primer identities which are passes and generating primers corresponding to those potential primer identities;

conducting an amplification process using those primers and performing a further evaluation on the results for one or more of those primers against one or more further criteria, the primers being deemed to pass or fail the further evaluation;

the pass primers forming a pool of primer candidates from which one or more primers for use in the amplification of DNA sequences are selected.

The evaluation may relate to the primer sequence and/or the primer length and/or the primer annealing temperature and/or the primer amplification efficiency and/or the balance of amplification between two or more primers for different single nucleotide polymorphisms.

The DNA sequence may be in a sample. The sample may be extracted from a collected source. The sample may be a mixture. One or more contributions to the sample may be analysed following amplification. The DNA sequence may be at least 20 bases long. Preferably the sequence incorporates one single nucleotide polymorphism.

The selecting of a single nucleotide polymorphism site may be made at random. Preferably the selection is made from one or more databases of single nucleotide polymorphism sites, for instance one or more databases accessible via the Internet. Preferably the chromosomal position of the SNP site is noted.

An initial on the potential single nucleotide polymorphism site may involve an evaluation of information known about that site or its surroundings. The surroundings may be the sequence on the 3′ or 5′ side of the single nucleotide polymorphism site. Preferably single nucleotide polymorphism sites are deemed to pass the evaluation if they or their surroundings are not coding regions and/or they or their surroundings are not known to be associated with coding regions and/or they or their surroundings are not diseased markers. Single nucleotide polymorphism for which they or their surroundings are coding regions, are known to be associated with coding regions or are diseased markers are preferably deemed to fail the evaluation. Preferably the sequence at least 50 bases, and more preferably at least 100 bases, on one or both sides of the SNP is considered for the purposes of passing or failing this initial evaluation.

Preferably only one potential primer identity is generated for each SNP site. The potential primer identity may be generated using computer software and/or manually. Preferably the potential primer identity has a nucleotide sequence which pairs to the nucleotide sequence up to and adjacent to the SNP site, preferably on the 3′ side.

The evaluation on the potential primer identity may involve an evaluation of its length and/or of its annealing temperature and/or of the bases from which it is formed. Preferably potential primer identities having a melting temperature, Tm, outside the range 55 to 65° C. and more preferably outside the range 58 to 62° C. are deemed to fail the evaluation. Preferably potential primer identities with melting temperatures, Tm, falling within the range 55 to 65° C. and more preferably the range 58 to 62° C., inclusive, are deemed to pass the evaluation. Preferably primers of between 15 and 35 bases and more preferably between 17 and 30 bases are deemed to pass the evaluation. Preferably potential primer identities of length outside these ranges are deemed to fail the evaluation. Preferably a potential primer identity which is formed of greater than 60% A and/or T bases, more preferably greater than 50% A or T bases, and ideally greater than 40% A or T bases, is deemed to fail the evaluation. Preferably potential primer identities with A and /or T compositions below one or more of these levels are deemed to pass the evaluation. Preferably single nucleotide polymorphism sites which include an adjacent sequence of 30 bases or less of which greater than 50% is formed of A or T bases is deemed to fail the evaluation. The adjacent sequence may be to the 3′ end side or the 5′ end side of the SNP site.

Preferably the selection and evaluation is repeated for a plurality of single nucleotide polymorphism sites and related potential primer identities. Preferably the selection and evaluation process is repeated until at least 5, more preferably at least 10 and ideally at least 15 passes of the evaluation have been obtained. Preferably each pass involves a single nucleotide polymorphism site and a single potential primer identity therefor.

The primers may be generated from the potential primer identities by conventional construction techniques. Preferably the primer sequences correspond identically to the potential primer identities.

Preferably the amplification process is a PCR based amplification. Preferably each of the pass primer is present in the amplification process. The amplification process may particularly be carried out according to the features, options and possibilities set out for such a process in WO01/07640, the contents of which are incorporated herein by reference.

Preferably the further evaluation is performed on each of the primers present in the amplification process. Preferably the same evaluation is performed on each of those primers. The further criteria may be whether or not the SNP site is monomorphic. A monomorphic SNP may be deemed a fail. A polymorphic SNP may be deemed a pass. The further criteria may be whether or not multiple copies of the SNP incorporating sequence are present on the genome. The presence of multiple copies may be deemed a fail. The presence of a single sequence of that sequence may be deemed a pass. The further criteria may be a level and/or efficiency and/or extent of amplification. An insufficient level and/or efficiency and/or extent may be deemed a fail. The further criteria may be whether or not artifacts are produced in the amplification process by the primer. The production of artifacts may be deemed a fail. The further criteria may be whether or not the allelic products produced are balanced. An unbalanced allelic product may be deemed a fail.

The pass primers may form a pool, in the form of a SNP site and associated primer/potential primer identity which has passed the evaluation and the further evaluation. The pass primers and/or their SNP sites may be the subject of a still further evaluation. The still further evaluation may involve considering the frequency of occurrence for each allele of the SNP site within the population as a whole, or within one or more subsets of the population. Preferably one or more subsets of the population are considered, ideally with at least 10 and more preferably at least 25 individuals in each of those subsets. Subsets for white Caucasian and/or Asian and/or Afro-Caribbean may be considered. Most preferably the frequency of occurrence of the alleles for the SNP site is considered against each of those population sub-groups. Preferably the still further evaluation gives rise to a pass or fail being deemed to occur. Preferably an SNP site is considered a fail if the frequency of occurrence of one or the alleles is outside the range 0.1 to 0.9 for the population and/or one or more of the population sub-groups. Preferably a fail is deemed to occur if the frequency of allele occurrence for any of the possible alleles is outside the range 0.1 to 0.9 for any of the population sub-groups. Preferably an SNP site is considered a pass if the frequency of occurrence of one or the alleles is inside the range 0.1 to 0.9, inclusive, for the population and/or one or more of the population sub-groups. Preferably a pass is deemed to occur if the frequency of allele occurrence for any of the possible alleles is inside the range 0.1 to 0.9, inclusive, for all of the population subgroups.

Preferably a plurality of the primers which pass the still further evaluation and/or which have passed the further evaluation are subjected to verification testing. Preferably the verification testing involves forming a mixture of primers including two or more of the pass primers, more preferably at least five of the pass primers, and ideally between 8 and 20 of the pass primers. Preferably the verification involves the use of the mixture in an amplification process, ideally an amplification process of the type set out in WO 01/07640.

The amplification may be carried out using a gel base and/or using a micro-array arrangement and/or using a solid support system.

Preferably the verification includes confirmation of the primers as having a melting temperature within a total spectrum of 2° C. of one another and/or primers all having lengths between 17 and 30 bases and/or primers having substantially equivalent amplification efficiencies and/or no artifact producing amplification occurring.

According to a second aspect of the invention we provide a method of producing a mixture of primers, the mixture being for use in the amplification of a plurality of DNA sequences each incorporating one or more single nucleotide polymorphisms, the method including:

selecting a single nucleotide polymorphism site;

generating at least one potential primer identity for amplifying the single nucleotide polymorphism site and performing an evaluation on the potential primer identity and/or the single nucleotide polymorphism site against one or more criteria, the potential primer identity and/or the single nucleotide polymorphism site being deemed to pass or fail the evaluation;

obtaining a plurality of single nucleotide polymorphism sites and related potential primer identities which are passes and generating primers corresponding to those potential primer identities;

conducting an amplification process using those primers and performing a further evaluation on the results for one or more of those primers against one or more further criteria, the primers being deemed to pass or fail the further evaluation;

the pass primers forming a pool of primer candidates and selecting one or more of the primers and producing a mixture of primers incorporating those one or more primers.

According to a third aspect of the invention we provide a method of amplifying a plurality of DNA sequences each incorporating one or more single nucleotide polymorphisms, the method including the use of a mixture of primers, one or more of the primers being selected for the mixture according to a method which includes:

selecting a single nucleotide polymorphism site;

generating at least one potential primer identity for amplifying the single nucleotide polymorphism site and performing an evaluation on the potential primer identity and/or the single nucleotide polymorphism site against one or more criteria, the potential primer identity and/or the single nucleotide polymorphism site being deemed to pass or fail the evaluation;

obtaining a plurality of single nucleotide polymorphism sites and related potential primer identities which are passes and generating primers corresponding to those potential primer identities;

conducting an amplification process using those primers and performing a further evaluation on the results for one or more of those primers against one or more further criteria, the primers being deemed to pass or fail the further evaluation;

the pass primers forming a pool of primer candidates and the one or more of the primers being selected form that pool.

The method of amplifying may be part of a method of investigating single nucleotide polymorphisms in a sample of DNA. The method of investigating may comprise contacting the DNA containing sample with at least one first set of primers, amplifying the DNA using those primers to give an amplified product, contacting at least a portion of the amplified product with at least one second set of primers, amplifying the DNA using those second set of primers to give a further amplified product and examining one or more characteristics of the further amplified product.

In one embodiment of the invention one or more, preferably all, of the first sets of primers may include two forward primers and a reverse primer. One or more, preferably all, of the first sets of primers may consist of two forward and a reverse primer. The forward primers and reverse primer preferably include sequences which anneal to the 3′ and 5′ sides respectively of the SNP at the locus incorporating the SNP under investigation.

In an alternative embodiment of the invention, one or more, preferably all of the first sets of primers may include a forward primer and a reverse primer. One or more, preferably all of the first sets of primers may consist of one forward primer and one reverse primer. The forward primer and reverse primer preferably include sequences which pair/anneal to the 3′ and 5′ sides respectively of the SNP at the locus incorporating the SNP under investigation.

The first set of primers may include one or more primers including a locus specific portion and a further portion. Preferably the forward primers are so provided. Preferably the further portion is attached to the 5′ end of the locus specific portion, particularly in the case of forward primers. The 3′ end of the forward primer is preferably provided with a SNP identifying portion. The further portion is preferably attached to the locus specific portion by a SNP related portion.

In one embodiment of the invention the locus specific portion preferably includes a sequence which matches the sequence of the locus sequence in the vicinity of the SNP under investigation. The match may occur at between 2 to 10 bases to the respective sides of the SNP under investigation. More preferably the sequence matches the locus sequence for the locus sequence adjacent to the SNP under investigation, ideally up to and including the nucleotide before the SNP on the 3′ side of the SNP. Preferably the forward primers of a first set of primers are provided with identical sequences for the locus specific portion.

In one embodiment of the invention the SNP identifying portion is preferably a single nucleotide. The SNP identifying portion may be a C for investigating an SNP where the SNP may be a G nucleotide. The SNP identifying portion may be a G nucleotide for investigating an SNP where the SNP may be a C nucleotide. The SNP identifying portion may be a T nucleotide for investigating an SNP where the SNP may be an A nucleotide. The SNP identifying portion may be an A nucleotide for investigating an SNP where the SNP may be a T nucleotide. Preferably the SNP identifying portion for one forward primer of a set is one of C or G or A or T, with the SNP identifying portion of the other forward primer of the set being one of C or G or A or T, but different from the SNP identifying portion of the first forward primer of the set. Preferably the SNP identifying portions are provided to target the two possible variations of the SNP in question, for instance C and T for the primers to investigate G or A for the SNP, C or G for the primers to investigate G or C for the SNP and so on.

Preferably the SNP identifying portion forms the 3′ end of the forward primers of the first set.

The further portion preferably includes a sequence which does not match the locus sequence on the locus's 3′ side of the locus sequence matching the locus specific portion of the primer. More preferably the sequence does not match the sequence of the locus in the vicinity of the SNP under investigation. Ideally the sequence does not anneal to, and particularly does not match, the sequence of any published part, ideally any part, of the entire DNA sequence of the entity from which the DNA containing the SNP under investigation was obtained, for instance Homo Sapiens. The inability of the sequence of the further portion to amplify human DNA is a particularly preferred feature. Preferably the forward primers of a first set of primers are provided with identical sequences for the further portion.

Preferably the further portion forms the 5′ end of the forward primers of the first set.

The further portion of two or more of the forward primers of the first set may have an equivalent sequence. All the forward primers of the first set may be provided with further portions of equivalent sequence.

In a preferred embodiment of the invention, the further portion of at least one of the forward primers of the first set is different from the further portion of at least one of the other forward primers of the first set, at least in part. Preferably the further portion of each forward primer of the first set is different from the further portion of each of the other forward primers of the first set, at least in part. It is preferred that the forward primers are different from one another with respect to at least 25% of the nucleotides forming the further portion of the forward primers. Differences in sequence, ranging between 25% and 100% of the nucleotides forming the further portion of the forward primers may be employed. The differences in sequence may form one or more distinguishing portions. One or more distinguishing portions may be provided as or within the further portion of the forward primers. A distinguishing portion may be provided at the 5′ end of the further portion of the forward primer. The distinguishing portion may be provided at the 3′ end of the further portion of the forward primer. Preferably the distinguishing portion is provided at an intermediate location within the sequence of the further portion. Preferably a 5′ end portion, distinguishing portion and 3′ end portion defines the further portion of the forward primers.

The further portion of one or more of the primers in the first set may be provided with one or more portions which correspond with one or more portions in the further portion of one or more of the other primers in the first set. The nucleotides of the further portion of one or more of the forward primers may be equivalent to the nucleotides of one of the other forward primers, outside the distinguishing portion of the further portion. In particular, the 5′ end portion and/or 3′ portion of the further portion of one or more of the forward primers may be equivalent to the corresponding further portion of one or more of the other forward primers. Preferably all of the forward primers are provided with equivalent 5′ end and/or 3′ end portions to one another. The equivalent portions may form between 1 and 25% of the sequence of the further portion of the primers. Preferably the equivalent portions form between 10 and 25% of the sequence of the further portions. The reverse primer or primers of the first set may be provided with equivalent portions too.

The SNP related portion is preferably a single nucleotide. The SNP related portion is preferably identical to the SNP identifying portion of that primer. Preferably the two forward primers are provided with SNP related portions which are identical with their respective SNP identifying portions. The SNP related portion may be a C for investigating an SNP where the SNP may be a G nucleotide. The SNP related portion may be a G nucleotide for investigating an SNP where the SNP may be a C nucleotide. The SNP related portion may be a T nucleotide for investigating an SNP where the SNP may be an A nucleotide. The SNP related portion may be an A nucleotide for investigating an SNP where the SNP may be a T nucleotide. Preferably the SNP related portion for one forward primer of a set may be one of C or G or A or T, with the SNP related portion of another primer of the set being one of C or G or A or T, but different to the SNP related portion of the first primer of the set. Preferably the SNP related portions for the primers of a set are provided to match the SNP identifying portion of their respective primers.

Preferably during amplification the SNP related portion results in the amplified copies of the locus incorporating the SNP having an SNP repeat introduced into them. Ideally, the repeat has a base identity identical to that of the SNP.

Preferably the locus specific portion and SNP identifying portion of one of the forward primers anneals to the 3′ side of the locus having the SNP under investigation. Preferably the locus specific portion and SNP identifying portion of another, ideally the other, of the forward primers does not anneal to the 3′ side of the SNP under investigation. Preferably the annealing primer anneals due to a match between the SNP identifying portion and the SNP site, (for instance C matching to G). Preferably the non-annealing primer does not anneal due to a mismatch between the SNP identifying portion and the SNP site, (for instance, T mismatching with T).

The SNP under investigation may be a location with variation between individuals of any two bases selected from C or G or A or T nucleotides. For instance, the SNP under investigation may be a location with variation between individuals of either a T or A nucleotide, T or C nucleotide, T or G nucleotide, A or C nucleotide, A or G nucleotide or C or G nucleotide. One possible variation may be investigated at one or more sites, with one or more other potential variations being investigated at one or more other sites.

Two or more SNP's may be investigated using a simultaneous first amplification and/or simultaneous second amplification and/or simultaneous examination of the one or more characteristic of the further amplified product. Preferably at least the first amplification and second amplification are conducted simultaneously for a plurality of SNP investigations. The number of SNP's investigated simultaneously in one or more stages of the process may be greater than 20, preferably greater than 25, more preferably greater than 50 and ideally greater than 100.

The sample may be a sample of DNA extracted from a collected source.

The sample may be contacted with the first primer set by mixing the sample and primers together.

The sample may be a mixture. One or more contributions to the sample may be analysed as the sample itself using the present invention. The mixed sample may include male and female DNA. One of the sexes of DNA, particularly the male, may be present in low concentrations relative to the other sex. For instance, the minor sex DNA contribution may form less than 1% of the sample, potentially less than 0.1% and even less than 0.05%. The sample may contain samples from two or more sources. The method may investigate the minor sample in a mixture from two or more sources. The minor sample may form less than 1% of the mixed sample, potentially less than 0.1% of the mixed sample and even less than 0.05% of the mixed sample.

The investigation may indicate the amount of DNA in a mixed sample from one or more of the sources. The indication may be based on a comparison of the experimentally determined results, for instance the level of a distinctive unit present, compared with a set of calibration results based on investigation of known amounts of DNA in a sample.

The first amplification is preferably performed by PCR. The amplification preferably involves between 18 to 60 cycles, more preferably 20 to 40 cycles.

The amplification cycles, particularly where the first and second amplification processes are used, may have the following characteristics. Preferably the amplification cycles include a first cycle set in which the annealing temperature of the cycle is similar or above the melting temperature of the first set of primers, particularly of the locus specific portion of the first set of primers and/or similar or above the second set of primer. The amplification cycles may include a second set of cycles, with preferably, the annealing temperature in the second set of cycles being similar or below the melting temperature of the first set of primers and/or above the melting temperature of the second set of primers. The melting temperature of the first set of primers may rise after one or two cycles. The amplification cycles may include a third set of cycles, with, preferably, the annealing temperature in the third set of cycles being below the melting temperature of the first set of primers and/or similar or above the melting temperature of the second set of primers.

It is preferred that the first set of cycles provide between 2 and 10 cycles. It is preferred that the second set of cycles provide between 3 and 15 cycles. It is preferred that the third set of cycles provide between 15 and 35 cycles. Preferably the total of cycles provided in the first, second and third sets does not exceed 40 cycles.

It is preferred that the denaturation temperature for the first and/or second and/or third set of cycles be 92 to 96° C., ideally 94° C.

It is preferred that the annealing temperature for the first and/or second and/or third set of cycles be between 60 and 62° C., ideally 61° C. It is preferred that the annealing temperature for the second set of cycles be between 70 and 78° C., ideally between 72 and 75° C.

It is preferred that the extension temperature for the first and/or second and/or third set of cycles be between 70 and 75° C., ideally 72° C.

Amplification preferably results in extension of the annealed forward primer from its 3′ end towards the 5′ end of the target sequence. Amplification preferably results in extension of the reverse primer from its 3′ end towards the 5′ end of its target sequence. Preferably further cycles of amplification result in extension of the forward primer sequence towards the 5′ end of its target, including the reverse primer sequence. Preferably further cycles of amplification result in extension of the reverse primer sequence towards the 5′ end of its target, including one or more or all of the forward primer sequence and particularly the SNP identifying portion, locus specific portion, SNP related portion and further portion.

A portion of the amplified product may be removed and contacted separately with the second set of primers. Contact with the second set of primers may occur in a separate vessel to the contact with the first set of primers. This is particularly preferred where universal primers incorporating molecular beacons are used. Preferably a two tube and/or branched PCR process is used where universal primers incorporating molecular beacons are employed.

The first and second amplifications may occur in the same vessel. The first and second amplifications may occur substantially simultaneously. Preferably the method includes adding one or more of the first set of primers and one or more of the second set of primers to the sample to be amplified prior to conducting amplification cycles.

The one or more first sets of primers may be provided at a concentration of between 20 and 80 nM, more preferably between 40 and 60 nM and ideally at 50 nM+/−5%. Preferably the primers which do not compete and/or for which site overlap does not occur are provided at these levels. Where primer competition could occur and/or where primer site overlap occurs preferably the primer's relative concentrations are balanced. The reverse primer concentration for such a simultaneous process may be between 75nM and 125nM, for instance 100nM+/−10%.

The second set of primers may be provided at a concentration of between 20 and 80 nM, more preferably between 40 and 60nM and ideally at 50nM+/−5%. The amount of the second set of primers added may be defined by Cn x L, where Cn is the concentration of the primers and L is the number of loci under consideration +/−2 and ideally is the number of loci under consideration, particularly where L is less than 100 or even less than 50. Preferably the maximum second set of primers concentration is 1000nM.

Particularly where the first and second sets of primers are present together, it is preferred to provide the second set of primers and first set of primers at a concentration ratio of at least 5:1. A ratio of at least 10:1, more preferably at least 20:1 and ideally at least 30:1, second set concentration: first set concentration may be provided. The first set may be provided at a concentration of between 5 and 400nM, more preferably between 10 and 200nM. The second set may be provided at a concentration of between 300nM and 5000nM, more preferably between 400 and 4000nM.

Particularly where the first and second sets of primers are present together, it is preferred to use an annealing temperature at which at least 80% of the second set of primers remain single stranded, more preferably a temperature at which at least 95% of the second set of primers remain single stranded and ideally a temperature at which at least 99% of the second set of primers remain single stranded, for some of the cycles of the amplification process. A lower annealing temperature may be used for other cycles of the amplification process. Preferably the higher temperature annealing is used at least in cycles 3 to 30, more preferably in cycles 3 to 40. A lower annealing temperature may be used in the first two cycles. A lower annealing temperature is preferably used in at least the last two cycles. The lower annealing temperature is preferably a temperature at which at least 80%, more preferably at least 90% and ideally at least 99% of the second set of primers anneal.

The amplified product may be contacted with the second primer set by mixing the sample and primers together.

The second set of primers may include one, two, three or four forward primers. A reverse primer may be present, but the second set of primers may lack a reverse primer.

The invention may only provide one second set of primers provided.

In one embodiment of the invention preferably the one second set of primers consisting of two forward primers and a reverse primer. One or more, preferably all, of the second sets of primers may include two forward primers and a reverse primer. One of the forward primers of the second set preferably includes a sequence which anneals to the SNP incorporating strand on the 3′ side of the SNP. The reverse primer of the second set preferably includes a sequence which anneals to the 3′ side of the base pairing to the SNP. More preferably one of the forward primers includes a sequence which anneals to the 3′ side of the SNP repeat. Preferably the other forward primer or primers does not anneal.

In the one embodiment of the invention the second set of primers may include one or more primers including a second further portion. Preferably the forward primers are so provided. Preferably the second further portion is provided with a second SNP identifying portion and/or more preferably an SNP repeat identifying portion. The second SNP or SNP repeat identifying portion may be attached to the 3′ end of the second further portion, particularly in the case of forward primers. The 5′ end of the forward primer is preferably provided with a distinctive unit.

In the one embodiment of the invention the second further portion preferably includes a sequence which pairs to the sequence of the amplified product in the vicinity of the SNP identifying portion and/or, more preferably, SNP repeat related portion thereof. More preferably the further portion sequence adjacent to the SNP related portion, ideally up to and including the nucleotide before the SNP related portion matches the sequence of the amplified product adjacent to the SNP repeat, ideally up to and including the nucleotide before the SNP repeat. Preferably the forward primers of a second set of primers are provided with identical sequences for the second further portions.

In an alternative embodiment of the invention it is preferred that the one second set of primers consists of one forward primer and one reverse primer. One or more, preferably all, of the second set of primers may consist of one forward primer and one reverse primer. Preferably the forward primer of the second set includes a sequence which anneals to the SNP incorporating strand on the 3′ side of the SNP. Preferably the reverse primer of the second set includes a sequence which anneals to the 3′ side of the base pairing to the SNP. Most preferably the forward primer includes a sequence which anneals to the sequence which pairs to the sequence produced by the copying of the further portion of the forward primer and/or which corresponds to the sequence of the further portion of the forward primer of the first set.

In the alternative embodiment of the invention, the second set of primers may include a primer including a second further portion and more preferably consisting of a second further portion. Preferably the forward primer is so provided. Preferably the second further portion is provided with a sequence which pairs to the sequence of the amplified product in the vicinity of the sequence which pairs to the further portion of the forward primer of the first set. More preferably, the second further portion includes a sequence which matches the sequence of the first further portion and/or pairs to the sequence of the amplified product matching the first further portion.

Preferably the sequence of the second further portion does not anneal to, and particularly does not match, the sequence of any published part, ideally any part, of the entire DNA sequence of the entity from which the DNA containing the SNP under investigation was obtained, for instance Homo Sapiens. The inability of the sequence of the second further portion to amplify human DNA is a particularly preferred feature. Preferably the forward primers of a second set of primers are provided with identical sequences for the second further portion.

In the one embodiment of the invention the second SNP related portion is preferably a single nucleotide or two nucleotides.

In the one embodiment of the invention preferably the second SNP related portion of one primer of the second set is, or includes, a nucleotide which is identical to the SNP identifying portion and/or SNP related portion of a primer of the first set. Preferably another, ideally the other, primer of the second set has a second SNP related portion which is, or includes, a nucleotide which is identical to the SNP identifying portion and/or SNP related portion of another, ideally the other, primer of the first set.

In the one embodiment of the invention where a single nucleotide forms the second SNP related portion, the second SNP related portion may be a C nucleotide when amplifying a target in which the SNP or SNP repeat is a G nucleotide. The second SNP related portion may be a G nucleotide when amplifying a target in which the SNP or SNP repeat is a C nucleotide. The second SNP related portion may be a T nucleotide when amplifying a target in which the SNP or SNP repeat is an A nucleotide. The second SNP related portion may be an A nucleotide when amplifying a target in which the SNP or SNP repeat is a T nucleotide. The second SNP related portion for one forward primer of a second set may be one of C or G or T or A with the second SNP related portion of another primer of the second set being one of C or G or A or T, but different to the second SNP related portion of the first primer of that set where the SNP or SNP repeat under investigation could be any two of C or G or T or A nucleotides.

In the one embodiment of the invention the second SNP related portion may be formed of two nucleotides. Preferably the end nucleotide of the two matches with the nucleotide of the SNP or SNP repeat of interest. Preferably the nucleotide adjacent to the end nucleotide of the second SNP related portion is a mismatch with the base adjacent to the SNP or SNP repeat in the target sequence.

In the one embodiment of the invention preferably the second SNP related portion forms the 3′ end of the forward primers of the second set.

An exonuclease digestion prevention unit may be provided towards the 3′ end of the forward primer or primers of the first and/or second set. The exonuclease digestion prevention unit may be phosphorothioate. The exonuclease digestion prevention unit may be provided at the end of the second further portion and/or the junction of the second further portion and second SNP related portion.

Preferably the second further portion and/or second SNP related portion of the forward primer and/or of one of the forward primers anneals to the 3′ side of the SNP or SNP repeat. Preferably the second further portion and/or second SNP related portion of another, ideally the other, of the forward primer and/or of the forward primers does not anneal to the 3′ side of the SNP and/or SNP repeat. In one embodiment of the invention preferably the annealing primer anneals due to a match between the second SNP related portion and the SNP repeat and/or adjacent sequences. Preferably the non-annealing primer does not anneal due to a mismatch between the second SNP related portion and the SNP repeat. In an alternative embodiment of the invention preferably the annealing primer anneals due to a match between the second further portion and a sequence which paired to the first further portion.

The second amplification is preferably performed by PCR. The amplification preferably involves between 18 and 30 cycles, more preferably 20 to 25 cycles.

One or more of the primers of the first and/or second set may be provided with one or more portions which are complimentary to one or more portions on one or more of the other primers in that set. The complimentary portion or portions are preferably provided in the further portion of the primers of the first set. The complimentary portion or portions are preferably provided in the second further portion of the primers of the second set. Preferably a complimentary portion is provided on each of the primers of a set. Preferably at least two complimentary portions are provided on each of the primers of a set. Preferably a complimentary portion is provided at the 3′ end of a primer, ideally all the primers. Preferably a complimentary portion is provided at the 5′ end of a primer, ideally all of the primers. Preferably the 3′ end complimentary portion of one primer is complimentary to the 5′ end complimentary portion of another primer, ideally all the other primers of the set and/or both sets. Preferably the 5′ end complimentary portion of one primer is complimentary to the 3′ end complimentary portion of another primer, ideally all the other primers of the set and/or both sets. A locus specific portion may be provided on the further portion including the complimentary portion or portions, particularly on the 3′ end. The further portion and/or second further portion may include a sequence matching the sequence of the locus under consideration, particularly provided between two complimentary portions. The complimentary portions may be at least 3 nucleotides long, more preferably between 3 and 20 nucleotides long. The complimentary portions are preferably both of the same length. The complimentary portions may form between 5 and 40% of the further portion and/or second further portion. One, two, three or four primers of a set may be provided in this way. Preferably the reverse primer or primers are similarly provided.

The further amplified product, or a portion thereof, may be removed from the vessel in which the amplification is performed to examine the one or more characteristics. Alternatively or additionally, the one or more characteristics may be examined with the further amplified product in the vessel in which amplification is performed.

The one or more characteristic of the further amplified product may be examined by means of the presence and/or absence of a distinctive unit in the further amplified product. The distinctive unit may be incorporated in the further amplified product or be associated there with. The distinctive unit may be introduced during the amplification process and/or in a subsequent step. The subsequent step may comprise hybridisation, for instance, of a component to the SNP base. The component may be a dideoxynucleotide, particularly a dideoxynucleotide incorporating a distinctive unit such as a dye.

The distinctive unit may be a dye label or colour producing molecule.

The distinctive unit may be a sequence of DNA, for instance a molecular beacon. The sequence of DNA, for instance a molecular beacon, may comprise a sequence of DNA incorporating a dye molecule. The sequence of DNA may be a single strand. The sequence of DNA may be looped by joining one part of the sequence to another. Preferably the dye molecule is in the loop, still more preferably in one part of the sequence which is joined to another. Preferably the dye molecule is in proximity with a quencher molecule. Preferably the quencher molecule prevents the dye molecule characteristic, for instance fluorescence, being visible. Preferably the dye molecule becomes visible, for instance fluorescent, upon activation. Preferably activation is caused by primer extension into the sequence of the molecular beacon. Activation preferably occurs through the opening of the loop. The molecular beacon sequence may be FACGCGCTCTCTTCTTCTTTTGCGCG-Q where F is a distinctive unit such as a dye, and Q is a quenching unit or vice versa. Preferably the parts of the molecular beacon sequence which join to one another are the stems ACGCGC from the 5′ end and GCGCG from the 3′ end. Preferably the universal primer incorporating molecular beacon does not contain phosphorothioate bonding. Preferably none of the second set of primers contain phosphorothioate bonding. Ideally none of the first or second primers contain phosphorothioate bonding. Where molecular beacons are used, the amplification product may be examined for one or more characteristics in the amplification reaction vessel. For instance, the Roche Light Cycler™ or other such instruments may be used for this purpose.

The distinctive unit may be visible under daylight or conventional lighting and/or may be fluorescent.

The distinctive unit may be an emitter of radiation, such as a characteristic isotope.

The distinctive unit is preferably provided at the 5′ end of one or the primers, more preferably on a forward primer and ideally with a different distinctive unit for the other forward primer of the second set.

Preferably the distinctive unit is indicative of the nucleotide present at the SNP. Preferably a different distinctive unit is present if one nucleotide is present at the SNP and than if the other nucleotide is present at the SNP. Different distinctive units may be provided for indicating the SNP at one locus when compared with the distinctive units for indicating the SNP present at a different locus.

The examination may involve separating the further amplified product relating to one SNP from the further amplified product from one or more other SNP's. Preferably the further amplified products for each SNP are separated from one another. Electrophoresis may be used to separate one or more of the further amplified products from one another. The further amplified products may be separated from one another based on size of the further amplified products, for instance due to the different length of the further amplified products.

The examination may involve analysing the response of the further amplified product, for instance in the vessel in which amplification was performed, to radiation of various wavelengths, for instance fluorescent light.

The examination may involve the use of micro-fabricated arrays.

The further amplified product may be contacted with one or more components retained on a solid support. One or more of the components may be an oligonucleotide, preferably with its 5′ end tethered to the support. Preferably the oligonucleotide has a sequence which pairs/anneals with the sequence of at least one, ideally only one, of the further amplified products.

In an embodiment the oligonucleotide may have a sequence which pairs/anneals with the sequence of at least one, ideally only one, of the further amplified products up to the base before the base which is the SNP site. Only a portion of the further amplified product may pair/anneal to the oligonucleotide. Preferably a particular further amplified product type pairs/anneals to a particular oligonucleotide.

In an another embodiment, the oligonucleotide may have a sequence which pairs/anneals with the sequence of at least one, ideally only one, of the further amplified products along the sequence corresponding to the locus specific portion and the further portion. Preferably the further portion of the further amplified product includes a distinctive unit. The distinctive unit is preferably a dye. Preferably a different dye is present on each different further amplified product.

A plurality of such components, such as a plurality of oligonucleotides may be provided. A plurality of different oligonucleotides may be provided with each having a sequence which pairs/anneals to a further amplified product, ideally only one such product. It is particularly preferred that each oligonucleotide type pairs/anneals to a different further amplified product type from the others. The plurality of different types of oligonucleotides may be provided at a plurality of different, ideally discrete locations on the support.

The solid support may be glass, silicon, plastics, magnetic beads or other materials.

In an embodiment one form of the invention, the oligonucleotide and paired/annealed further amplified product may be contacted with one or more further components. Preferably one or more of the further components includes a dideoxynucleotide. Preferably one or more of the further components includes a distinctive unit, such as a dye. Preferably different further component types include different distinctive units. Two or more components comprising two or more different dideoxynucleotides with a different distinctive unit attached to each may be provided. The dideoxynucleotides may be A, T, C or G. Three or four dideoxynucleotides may be provided, preferably each with a different distinctive unit.

One or more, preferably only one of the further components may selectively attach to the SNP base and/or 3′ end of the oligonucleotide. Preferably the selectivity of the attachment is based on the pairing of part of the further component's identity with the SNP base identity, such as the pairing of the dideoxynucleotide identity with the SNP base identity. Preferably the pairing incorporates the distinctive unit in the structure. Preferably the pairing incorporates the distinctive unit in the structure. Preferably non-pairing further components and their distinctive units are not incorporated in the structure.

The identity of the distinctive unit attached to the component in the structure is preferably investigated. Preferably the identity of the further component and/or the identity of the SNP is derived from the identity of the distinctive unit.

In another form of the invention, the oligonucleotide and paired/annealed further amplified product may be contacted with one or more additional components. The one or more additional components may be one or more further oligonucleotides. Preferably one or more of the additional components includes an end base, preferably at its 5′ end. Preferably one or more of the additional components includes a distinctive unit, such as a dye. Preferably different additional component types include different distinctive units. The additional components may comprise two or more different further oligonucleotides with a different distinctive unit and/or end base attached to each. The end base of the further oligonucleotides may be C, G, A or T. Three of four further oligonucleotides may be provided, preferably each having a different distinct unit and/or end base.

One or more, preferably only one, of the further oligonucleotides may selectively attach to the SNP base and/or 3′ end of the tethered oligonucleotide. Preferably the selectivity is based on the pairing of the further oligonucleotide's end base identity with the SNP base identity. Ligase may be provided in contact with the tethered oligonucleotide and/or further oligonucleotide and/or further amplified product. Preferably ligation occurs where the SNP base and end base pair, thereby incorporating the distinctive unit in the structure. Preferably non-pairing further components and the distinctive units are not incorporated in the structure.

The identity of the distinctive unit attached to the component in the structure is preferably investigated. Preferably the identity of the additional component and/or the identity of the end base of the additional component and/or the identity of the SNP is derived from the identity of the distinctive unit.

In yet another embodiment of the invention the further amplified product may incorporate an attachment unit. Preferably the attachment unit facilitates attachment of the further amplified product to a solid support. The solid support may be glass, silicone, plastics, magnetic beads or other materials. Preferably attachment is affected by means of a covalent bond. The attachment unit may be an amino group, preferably an amino group provided at the 5′ end of the further amplified product. It is preferred that the solid support is an epoxy-silane treated support in such cases. The attachment unit may be a phosphorothiate unit, ideally provided at the 5′ end of the further amplified product. In such a case, attachment to a bromo-acetomide treated solid support is preferred.

The further amplified product, attached to a solid support, is preferably contacted with one or more probes preferably having a different sequence from one another, at least in part. Preferably each probe has a common sequence portion to each other probe. It is particularly preferred that this common sequence portion correspond in sequence to the locus specific portion of the further amplified product. Preferably the probes incorporate at least one different sequence portion compared with one another. Preferably the different portions, for at least one of the probes, corresponds to the universal primer portion sequence of the further amplified product. It is preferred that contact of the probes with the further amplified product results in hybridisation of one of the probes to the further amplified product, ideally with no hybridisation of the other probe or probes. Preferably each probe has a distinctive unit attached, such as a dye unit. Preferably different distinctive units are used for each different probe.

The sample may be compared with another sample. The comparison may be based on comparing one or more of the one or more characteristic of the further amplified products for each sample. The samples may be compared to confirm a match in the characteristic between the samples. The samples may be compared to eliminate a match in the characteristic between the samples. The occurrence of the one or more further characteristic for one or more SNP's may be compared with information on the frequency of occurrence of the one or more further characteristic for the one or more SNP's in a population. The population may be a representative sample of the population of a country, an ethnic group or database.

The second and/or third aspects of the invention may include any of the features, options or possibilities set out herein, including those set out above in relation to the first aspect of the invention.

Various embodiments of the invention will now be described, by way of example only, and with reference to the accompanying drawings in which:

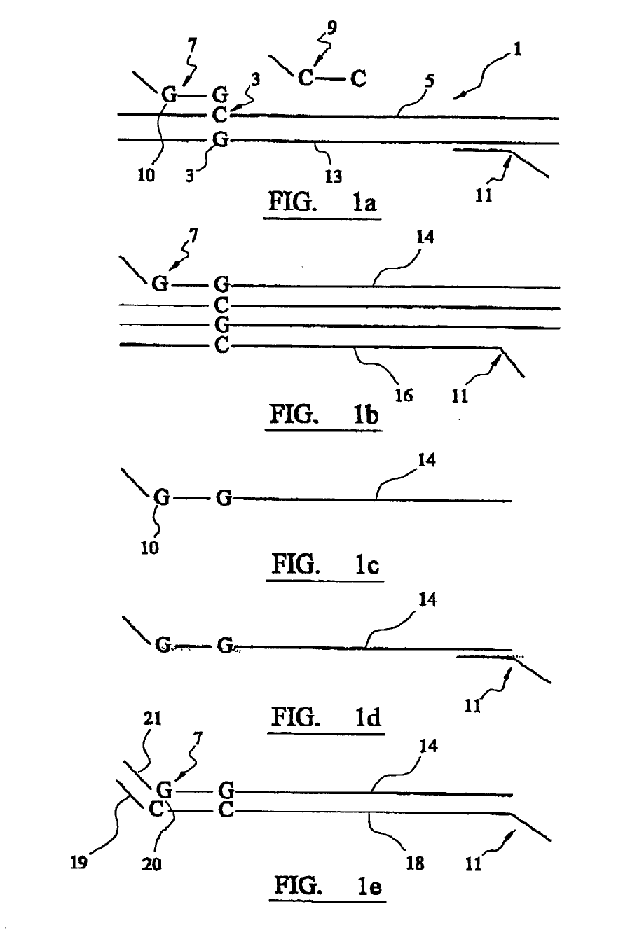

FIGS. 1a to 1e illustrate the various parts of the first stage of a process in which the present invention can be utilised;

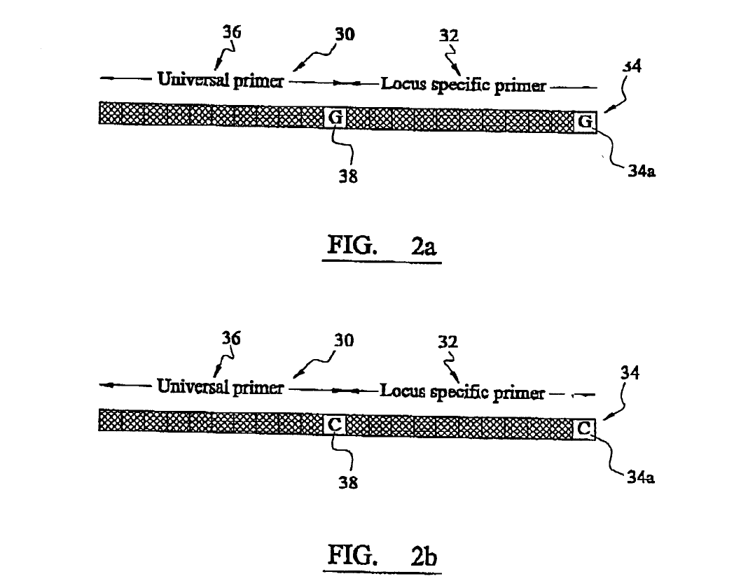

FIG. 2a illustrates one forward primer suitable for use in that technique;

FIG. 2b illustrates a second forward primer suitable for use in that technique and intended for use with the primer of FIG. 2a;

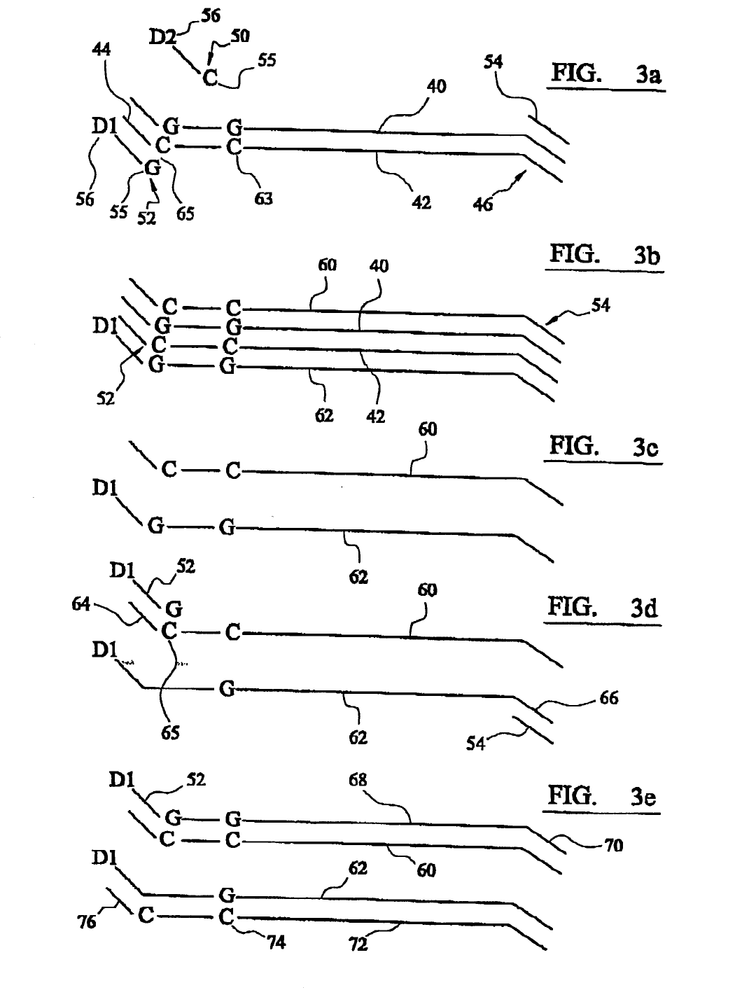

FIGS. 3a to 3e illustrate the various parts of the second stage of a process suitable for incorporating the present invention;

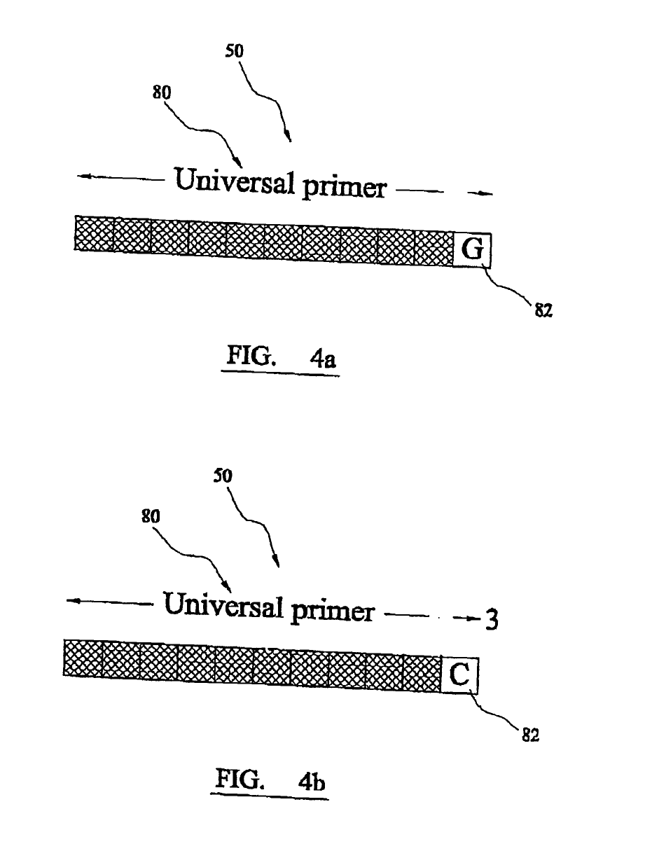

FIG. 4a illustrates a universal forward primer suitable for use in the second stage of the technique; and

FIG. 4b illustrates a second universal forward primer for use in the second stage of the technique and intended for use with the primer of FIG. 4a.

The nucleotide sequence of humans and other biological entities is in a large part consistent between individuals. Locations are known, however, at which variation occurs. One such form of variation is known as single nucleotide polymorphisms or bi-alleic markers, where the identity of a single nucleotide at a specific location is one of four possibilities from any of the four bases available, A, T, G or C. In many cases the variation is only bi-allelic and hence only one or two possibilities applies. Thus, some individuals may have a sequence incorporating a C base at a particular position, whereas other individuals will have a G base at that position; the surrounding sequences for both individuals being identical.

Medical diagnostics, forensic investigations and other DNA tracing applications make use of such single nucleotide polymorphisms (SNPs) for identification purposes. As the variation between individuals can only be between one of two options, a very substantial number of such locations, loci, must be considered for a statistically significant result, for instance the statistical significance of a match between a collected sample and an individual's makeup to be obtained, particularly in forensic applications. In medical applications very rare SNPs are selected and hence are more informative in limited numbers.

Investigating such a large number of loci, frequently several hundred, on an individual basis is extremely time consuming. To reduce the time taken, it might be desirable to construct multiplexes which allow a substantial number of loci to be investigated simultaneously based on PCR or other amplifying techniques. The design of reliable constructs for a large number of loci, however, is extremely difficult due to problems in interactions between the primers needed for the different loci, different conditions for suitably efficiency amplification of the different primers and a variety of other issues.

The technique of the present invention aims to select SNPs for investigation, provide primers for those SNPs and generate multiplexes from those primers while minimising the time and cost consuming investigations involved in the selection of those targets, the developments of the primers for those targets and the determination of appropriate multiplexes from such primers.

The technique is described in relation to the SNP investigating technique described in WO01/07640 published on Feb. 1, 2001, in particular (the contents of which are incorporated herein by reference), but is applicable to any SNP investigating technique which uses primers and particularly multiplexes of such primers to achieve rapid and cost-effective investigations.

Selection Process

As a first step a single SNP is chosen at random. In particular the choice is made from the entries in one of a number of databases of SNPs which are publically available on the Internet. In the SNP consortium database for instance, over a million SNPs are logged on to the database and are freely available to the public. The SNPs logged on this database, however, are unproven SNPs and in almost all cases have no attached information concerning their significance, medical or otherwise.

Having selected the SNP at random, the next step is to review the known information about that SNP and its surrounding sequence. Thus if the SNP or surrounding sequence is known to involve a coding region, a region known to be associated with coding regions, or is known to be a diseased marker, then that SNP is discarded. Once this consideration has been made it is determined whether or not the SNP and its surrounding sequence is fundamentally suited to the design of a primer for it. Using the technique set out in WO01/07640, for instance, the primer must abut the SNP site itself. A number of techniques are available for making this investigation, including commercially available software such as the Primer Express which is available from Applied Biosystems. In general, the determination includes a determination of the melting temperature of a primer which abuts that site, the length of primer necessary to anneal to that site effectively and the balance between the two. For forensic purposes, the applicants has determined that a melting temperature, Tm, of around 60° C. and primer lengths of around 20 bases are preferred. The determination may also include an evaluation of how AT rich the sequence is around the SNP site, as such sequences necessitate very long primers to give effective annealing.

If the determination suggests that the SNP is unsuited to investigation using primers of the desired length and melting temperature, then that SNP site is discarded and the process recommences with a selection of a further SNP at random from the public databases. Problems may occur where the SNP site has an adjacent sequence which has an A and/or T base make-up which is greater than 50% (as such sequences give poor annealing, and hence require longer primers and/or lower annealing temperatures) and/or where the primer length is in excess of 30 bases to achieve the desired extent of annealing and an annealing temperature of around 60° C. GC rich sequences are preferred.

Once an SNP is accepted as being fundamentally suited, then a forward and reverse primer sequence of around 20 bases in length is proposed, although variation of between 17 and 30 bases can be tolerated. The primer sequence is arrived at by providing a forward primer sequence which matches the DNA sequence adjacent to the SNP site, most preferably to the 3′ end side. The reverse primer sequence preferably matches the DNA sequence of the other strand at a site commencing within 2 to 120 bases of the 3′ end side.

This process is repeated until between 10 and 20 candidate SNPs and their relevant primers have been obtained.

A particular advantage of the technique described in WO01/07640 is that it enables multiplexes to be produced rapidly and evaluated, details of the general techniques provided in WO01/07640 are provided below under the heading “Amplification Process”, thereby providing the next set of information on the candidates.

The results obtained from the amplification process are informative in indicating those SNPs which in practice are inappropriate targets due to their properties and/or due to the particular primers selected to investigate them. Problems with the SNP may arise because they are mono-morphic and/or because multiple copies of the SNP and its surrounding sequence are present on the genome. Problems with the particular primer design initially selected may give rise to poor amplification, the amplification of artifacts or unbalanced efficiencies of amplification.

In medical applications, because the number of informative SNPs that are known is quite small, and because each of those SNPs is highly significant in its own right, from a diagnosis angle, substantial efforts tend to be made to re-design the primers individually, and/or in combination to address problems of these types. This is a time and cost consuming process. On the contrary, for the present forensic investigations, therefore, no primer re-design is entered into. Instead, those SNPs are discarded and only the successful SNPs proceed forward to further consideration.

The preparation of multiple multiplexes each investigating 10 to 20 candidates, enables a substantial number of promising candidates to be obtained and put forward to a further consideration. Some multiplexes may produce no such candidates, some may produce a number.

The next process involves the consideration of the SNP polymorphism itself against the actual variation for that SNP polymorphism in the population. In practice this consideration involves consideration against one or more subsets of the population. In this example this involves analysing 30 individuals from the white Caucasian, Asian and Afro-Caribbean ethnic groups to determine the proportion of each allele within that group. An SNP polymorphism continues to be considered as a suitable candidate provided the frequency of an allele is between 0.1 and 0.9 in each of the three ethnic groups considered. Completely contrary to medical investigations all unusual and absolutely all rare SNP polymorphisms are discounted from further consideration to maximise discriminating power.

Following this review and selection process a number of SNP polymorphisms and particular primers for investigating them are obtained. These candidates are then considered and a final selection for the multiplex of around 10 SNPs is made by selecting the best balance of melting temperature (with a couple of degrees of one another), amplification efficiency and balance in amplification between different primers.

Confirmatory tests using the multiplex are then run using the amplification technique described in more detail below.

The multiplex is suited to gel based analysis, but is also suited to micro-arrays and solid support systems.

The results of the method are the generation of a multiplex in a quick and cost effective manner which is forensically powerful, and yet is balanced in terms of the amplification performance.

Amplification Process

The process is based around two amplification stages, generally achieved through PCR, with both of the stages offering specifity in terms of the SNPs identified and amplified. The two amplification stages can be conducted separately or simultaneously and the amplification products can be analysed in a variety of ways.

FIG. 1 illustrates, according to one embodiment of the process, a series of stages involved in the first amplification process based around a target template 1 with a potential C or G single nucleotide polymorphism 3 in one strand 5 of that target template 1. As illustrated in step A, the target template strand 5 of the particular individual under consideration has a C nucleotide at the SNP site 3.

The first step in this amplification stage involves contacting the template target 1 with two different forward primers 7 and 9, and a reverse primer 11. The forward primers 7 and 9 are locus specific primers, described in more detail below.

Forward locus specific primer 7 is terminated by a G nucleotide thus rendering it a match with the C nucleotide at the SNP site 3 and resulting in annealing of that primer 7 with the strand 5. The reverse primer 11 is non-specific and anneals to the other strand 13 of the template 1 at the appropriate location.

In step B, the specific forward primer 7 and the reverse primer 11 extend to produce the strands 14 and 16 through primer extension.

Denaturation of the strands results in the separation of the strands 5, 13 from their respective copied strands 14 and 16. The copied strand 14 only is shown in step C and the illustration of the subsequent steps.

Subsequent primer annealing, step D, is then performed again using the two forward primers 7, 9 and reverse primer 11. As we are considering strand 14 it is the reverse primer 11 which attaches to the strand 14 due to its sequence. The specific forward primer 7 would attach to strand 16, once again annealing in alignment with the site of the SNP 3 in that strand's sequence, not shown.

In subsequent primer extension, stage E, the reverse primer 11 extends the sequence of new strand 18 with the appropriate sequence given the sequence of strand 14, including the extension to produce tail portion 19 which arose as the strand 14 included the tail portion 21 of the forward specific primer 7. Due to the G base in the sequence of strand 14, the new strand 18 includes an opposing C base so as to match the identity of the SNP at site 3 in original strand 5. Due to the G base in the sequence of strand 14, due to the SNP related base 10, the new strand 18 includes an opposing C base 20 so as to match the identity of the SNP related site 10 in the originally copied strand 14.

Repetition of steps A through E over 20 to 25 cycles produces many millions of copies of sequences incorporating the same SNP identity, SNP repeat and surrounding sequence as the target template 1.

FIG. 2a and b illustrate two locus specific forward primers, suitable for use in the stage detailed above, for use in investigating an SNP which could be either G or C. Each of the locus specific forward primers 30 consists of a locus specific portion 32 which has a sequence corresponding to the sequence of the loci under consideration up to the SNP site. The 3′ end 34 of the locus specific forward primers ends in a G nucleotide 34a for one of the primers, FIG. 2a, and in a C nucleotide 34b for the other primer, FIG. 2b. Due to this different nucleotide used at the position corresponding to the SNP, then depending upon the identify of the SNP actually encountered, one of the locus specific forward primers will anneal thereto, but the other will not. Thus it is the forward primer of FIG. 2a which anneals to the target in the example of FIG. 1. This selectivity in annealing gives consequential specifity in the subsequent amplification cycles of the first stage.

In addition to the locus specific portion 32 the locus specific forward primer 30 includes a “universal” primer portion 36. The “universal” primer portion 36 consists of a nucleotide sequence which is identical for each of the two loci specific forward primers, save for a single nucleotide location 38 at the junction between the universal primer portion 36 and loci specific portion 32 of the primer 30. The nucleotide at the location 38 is identical to the 3′ end nucleotide 34 of the locus specific portion 32 of the respective primer 30. Thus, the “universal” primer of FIG. 2a incorporates G in its sequence at location 38 to reflect the G nucleotide present at the 3′ end 34. The “universal” primer portion of FIG. 2b, on the other hand, includes a C at location 38 to reflect the fact that a C nucleotide forms the 3′ end 34 of this primer 30.

Whilst it is the locus specific portion 32 of the forward primers 30 which determines whether a primer anneals or not to the target, in the second and subsequent copying stages of the amplification process of stage 1, primer extension causes copying of the “universal” primer portion 36 of the primer sequence also and hence copying of the SNP equivalent nucleotide identity at location 38 too.

What is described above in relation to locus specific primer 7, 9, which incorporate SNP rated basis 10, the technique is more preferably applied using locus specific primer 7, 9 formed of a locus specific primer portion and a universal primer portion without a linking SNP related base. It should be noted in such cases that the universal primer portion has a nucleotide sequence which is different between each of the two forward primers, and that the variation in the sequence of the amplification products which is used in subsequent identification arises from this difference.

As previously stated the amplification process of the first stage results in a large number of copy sequences, including the SNP identity reflecting nucleotide and the matching nucleotide at location 38.

In the second stage of amplification, illustrated in FIG. 3, a further specific amplification process is performed. It is much preferred that the second stage of amplification be conducted in the same vessel as the first, substantially simultaneous with the first amplification process. Such a possibility is described in more detail below.

For this stage, an aliquot of the amplification products from the first stage, described above, are taken and contacted with a pair of “universal” forward primers and a “universal” reverse primer. These “universal” primers are described in more detail below.

In step A, the strands 40 and 42 (copy strands which are equivalent to strands 14, 18 produced in the first stage as illustrated above) produced by the first stage 1 are denaturated and contacted with the two “universal” forward primers 50, 52 and reverse “universal” primer 54.

The two “universal” forward primers differ in terms of the 3′ terminal end nucleotide 55 and in terms of a dye unit D or other form of label provided on the 5′ end 56. The 3′ end nucleotide 55 for the forward “universal” primers in this example is either C, “universal” primer 50, or G, “universal” primer 52.

As the strands 40 and 42 represent the outcome of copies of copies of the originals being taken, unlike strands 14, 18, they both have tail portions 44, 46 respectively which arise from the copying of the “universal” primer portions of the locus specific forward primer and reverse primer in the first stage.

The “universal” primers 50, 52 each have a sequence corresponding to the “universal” primer portion 34 of the first stage locus specific primers 30 up to location 38 of the locus specific forward primers 30. At location 55 the forward primers 50, 52 of the second stage have a base corresponding in identity to the identity of the nucleotide paring to the SNP repeat in the stage 1 process, in one case, and in the other case corresponding to the identity of the other option for the SNP repeat. The nucleotide identity for the “universal” primers 50, 52 at location 55, corresponding to location 38, is thus different for the two primers 50, 52, with one providing one of the options and the other providing the other.

In the illustrated example, primer 50 carries a C and primer 52 carries a G nucleotide at position 55.

The sequence of the primers 50, 52 and particularly the identity at position 55 determines whether or not that primer 50, 52 anneals to the tail portion 44 of the strand 42 or not. In the illustrated case, strand 42 carries the SNP nucleotide C at site 63 as this was a copy of the identity of the SNP at site 3 in the original target strand 5. The C identity is also repeated in the tail portion 44 at site 65 as this was copied due to the copying of the tail of the original primer 7 by the reverse primer 11 in the first stage. Asa consequence the sequence of the tail portion 44 of strand 42 provides an annealing site for “universal” primer 52, but not primer 50. The reverse primer 54 anneals to the tail portion 46 of strand 40 due to the sequence matching.

In alternative, preferred techniques, the sequence of the primers 50, 52 possesses the dye unit D, in the form of dye unit D1 or different dye unit D2, or other form of label, but lacks the 3′ end nucleotide identified in the preceding paragraphs. In this case, the differences in the tail portion sequence is due to the different universal primer portion sequences of the two different primers of the first stage give rise to the annealing of the primer where a match occurs, but not in the other case. As a consequence the dye unit D or other identity indicating label is introduced. In a still further alternative technique the dye unit D or other form of label is omitted from both the universal forward primers of the second stage, and the determination of the identities is achieved in a third stage as illustrated in FIGS. 12a through 12b, for example, of WO01/07640. Again, this is the matching versus non-matching universal primer sequences which are important in that situation.

Primer extension, step B, results in the production of strand 60 by matching strand 40, including SNP site copy C, and in the production of strand 62, including the match for the SNP, G, by matching strand 42 by the “universal” reverse primer 54 and specific “universal” forward primer 52 respectively. The SNP repeats are also copied.

Thermal denaturation is then used to separate the strands, step C, and from here on strands 60 and 62 only are considered although similar processes apply to the other strands too.

In annealing step D, the specific “universal” forward primer 52 anneals to the tail 64 of strand 60 due to the presence of a C nucleotide at the relevant position 65 in strand 60 and the consequential pairing to the “universal” forward primer 52. The reverse primer 54 anneals to the tail portion 66 of the strand 62.

In the further extension step E, the forward primer 52, which brings with it the label D1, extends the sequence of new strand 68, including tail portion 70. The reverse primer 54 extends the sequence of new strand 72, (thereby reproducing the SNP identity at site 74), including tail portion 76, (thereby reproducing the nucleotide corresponding to the SNP repeat 75 in that part too). Strand 62 already incorporates the label D1 from its start as the primer 52 in step A Once again, repeating stages A to E gives substantial amplification of the sequences and produces a great number of sequences label with a dye D1, the dye being selectively taken up as only one primer anneals and thus takes the dye into the sequence with it.

As described above, the second stage of the process uses a pair of “universal” primers on their own, illustrated in FIGS. 4a and 4b. These consist of a portion 80 having a sequence identical with the “universal” primer portion 32 of the locus specific primers 30 up to the single nucleotide variation at the end of the “universal” primer portion 32. The ends 82 of the universal primers of FIGS. 4a and 4b are different from one another and have an identity consistent with one of the two SNP possibilities, as is the case for the primers of FIGS. 2a and 2b. Thus, one “universal” primer 52, FIG. 4a, is provided with G at its terminal 3′ end 82 and the other “universal” primer 50, FIG. 4b, is provided with C at its terminal 3′ end 82.

During stage 2 of the process, these “universal” primers will selectively anneal to the amplification products of the first stage depending upon whether the tail portions extended and amplified during that stage incorporates the G or C variation.

Of course, equivalent primer types could be used with T or A variations in the above mentioned processes to investigate an SNP having potential T or A variation.

In the case of the alternative techniques, universal primers will selectively anneal to the amplification products in a first stage depending upon whether the tail portions have a sequence matching to the first universal primer, or second universal primer.

The different “universal” forward primers are provided with different labels/markers, in this case a JOE dye label and an FAM dye label respectively. The dye labels are provided at the 5′ end of the forward primer in the second stage of the process. Of course, other different dyes and other forms of marking, such as radio nudides could be used.

The “universal” primers were carefully designed to give desirable characteristics in terms of their melting temperatures, particularly a melting temperature of around 60° C. The sequences were also checked to ensure minimal hairpin formation and checked for minimal primer dimer formation. The sequences were also checked against human DNA sequence records and/or samples to ensure that human DNA is not amplified and to avoid any correspondence to any published sequence and particularly any part of the human DNA sequence. Primer dimer formation was also taken into account so as to keep such formation minimal.

1. A method of evaluating primers, the primers being for use in the amplification of DNA sequences incorporating one or more single nucleotide polymorphisms, the method including:
selecting a single nucleotide polymorphism site;
generating at least one potential primer identity for amplifying the single nucleotide polymorphism site and performing an evaluation on the potential primer identity and/or the single nucleotide polymorphism site against one or more criteria, the potential primer identity and/or the single nucleotide polymorphism site being deemed to pass or fail the evaluation;
obtaining a plurality of single nucleotide polymorphism sites and related potential primer identities which are passes and generating primers corresponding to those potential primer identities;
conducting an amplification process using those primers and performing a further evaluation on the results for one or more of those primers against one or more further criteria, the primers being deemed to pass or fail the further evaluation;
the pass primers forming a pool of primer candidates from which one or more primers for use in the amplification of DNA sequences are selected.

2. A method according to claim 1 in which the selecting of a single nucleotide polymorphism site is made at random.

3. A method according to claim 1 in which only one potential primer identity is generated for each SNP site.

4. A method according to claim 1 in which the evaluation on the potential primer identity involves an evaluation of its length and/or of its annealing temperature and/or of the bases from which it is formed.

5. A method according to claim 1 in which potential primer identities having a melting temperature, Tm, outside the range 58 to 62° C. are deemed to fail the evaluation.

6. A method according to claim 1 in which primers of between 17 and 30 bases are deemed to pass the evaluation.

7. A method according to claim 1 in which a potential primer identity which is formed of greater than 40% A or T bases, is deemed to fail the evaluation.

8. A method according to claim 1 in which single nucleotide polymorphism sites are deemed to pass the evaluation if they or their surroundings are not coding regions and/or they or their surroundings are not known to be associated with coding regions and/or they or their surroundings are not diseased markers.

9. A method according to claim 1 in which the selection and evaluation is repeated for a plurality of single nucleotide polymorphism sites and its related potential primer identity until at least 10 passes of the evaluation have been obtained.

10. A method according to claim 1 in which the further evaluation is performed on each of the primers present in the amplification process, the further criteria being whether or not the SNP site is monomorphic and/or whether or not multiple copies of the SNP incorporating sequence are present on the genome and/or the level and/or efficiency and/or extent of amplification and/or whether or not artifacts are produced in the amplification process by the primer and/or whether or not the allelic products produced are balanced.

11. A method according to claim 1 in which the pass primers form a pool, in the form of a SNP site and associated primer/potential primer identity which has passed the evaluation and the further evaluation and the pass primers and/or their SNP sites are the subject of a still further evaluation.

12. A method according to claim 11 in which the still further evaluation involves considering the frequency of occurrence for each allele of the SNP site within the population as a whole, or within one or more subsets of the population.

13. A method according to claim 11 in which an SNP site is considered a fail if the frequency of occurrence of one or the alleles is outside the range 0.1 to 0.9 for the population and/or one or more of the population sub-groups.

14. A method according to claim 1 in which a plurality of the primers which pass the still further evaluation and/or which have passed the further evaluation are subjected to verification testing, the verification testing involving forming a mixture of primers including at least five of the pass primers, and using the mixture in an amplification process.

15. A method according to claim 14 in which the verification includes confirmation of the primers as having a melting temperature within a total spectrum of 2° C. of one another and/or primers all having lengths between 17 and 30 bases and/or primers having substantially equivalent amplification efficiencies and/or no artifact producing amplification occurring.

16. A method of producing a mixture of primers, the mixture being for use in the amplification of a plurality of DNA sequences each incorporating one or more single nucleotide polymorphisms, the method including:
selecting a single nucleotide polymorphism site;
generating at least one potential primer identity for amplifying the single nucleotide polymorphism site and performing an evaluation on the potential primer identity and/or the single nucleotide polymorphism site against one or more criteria, the potential primer identity and/or the single nucleotide polymorphism site being deemed to pass or fail the evaluation;
obtaining a plurality of single nucleotide polymorphism sites and related potential primer identities which are passes and generating primers corresponding to those potential primer identities;
conducting an amplification process using those primers and performing a further evaluation on the results for one or more of those primers against one or more further criteria, the primers being deemed to pass or fail the further evaluation;
the pass primers forming a pool of primer candidates and selecting one or more of the primers and producing a mixture of primers incorporating those one or more primers.

17. A method of amplifying a plurality of DNA sequences each incorporating one or more single nucleotide polymorphisms, the method including the use of a mixture of primers, one or more of the primers being selected for the mixture according to a method which includes:
selecting a single nucleotide polymorphism site;
generating at least one potential primer identity for amplifying the single nucleotide polymorphism site and performing an evaluation on the potential primer identity and/or the single nucleotide polymorphism site against one or more criteria, the potential primer identity and/or the single nucleotide polymorphism site being deemed to pass or fail the evaluation;
obtaining a plurality of single nucleotide polymorphism sites and related potential primer identities which are passes and generating primers corresponding to those potential primer identities;
conducting an amplification process using those primers and performing a further evaluation on the results for one or more of those primers against one or more further criteria, the primers being deemed to pass or fail the further evaluation;
the pass primers forming a pool of primer candidates and the one or more of the primers being selected form that pool.
